(19) United States
(12) Patent Application Publication
(10) Pub. No.: US 2007/0027359 A1
(43) Pub. Date: Feb. 1, 2007

(54) Improved Control System for Supplying Fluid Medium to Endoscope

(75) Inventor: Golan SALMAN, Tirat Carmel (IL)

Correspondence Address: BRUCE E. LILLING;LILLING & LILLING P.C., P.O. BOX 560, GOLDEN BRIDGE, NY, 10526, US

(73) Assignee: STRYKER GI LTD., Haifa (IL)

(21) Appl. No.: 11/460,559

(22) Filed: Jul. 27, 2006

Related U.S. Application Data
(60) Provisional application No. 60/703,200, filed on Jul. 28, 2005.

(51) Int. Cl. A61B 1/00; A61B 1/12

(52) U.S. Cl. 600/121; 600/158; 600/159

(57) ABSTRACT

A control system for supplying a fluid medium to an endoscopic apparatus is disclosed. The apparatus comprises an operation handle and an insertion member insertable within a body channel, wherein there is provided a protection sleeve adapted to cover at least a portion of the insertion member. The control system comprises a system control unit with a pump for supplying compressed air at least to the channel for inflating the sleeve. The system control unit is provided with a duct, which is in fluid communication with the pump. The duct comprises a first branch through which the compressed air from the pump passes to the sleeve and a second branch having exit to atmosphere, said second branch is provided with a controllable flow regulating means.

### FIELD OF THE INVENTION

The present invention relates generally to the field of endoscopy and specifically to endoscopic apparatus used for colonoscopic procedures during which a flexible tube is inserted into the rectum and colon for examination of the colon interior for abnormalities. More particularly, the present invention refers to a control system for supplying to the endoscope a fluid medium, e.g. air, water etc.

### BACKGROUND OF THE INVENTION

There are known endoscopes employing inflatable flexible sleeves for propulsion of the endoscope within the colon.

Voloshin (U.S. Pat. No. 6,485,409) discloses an endoscope, which comprises an endoscopic probe, bending section for directing the probe within the colon (steering unit), insertion tube and a flexible covering sleeve or a sheath, which is coupled proximally to the probe. The sleeve is attached to the endoscope in such a manner that its folded section is retained between a cap and an internal spindle, which are located at the rear part of the probe. When inflated, the folded section unfolds over a flange of the internal spindle and an inner portion of the sleeve is pulled behind the steering unit in a distal direction.

Eizenfeld (WO 2004/016299; international application PCT/IL2003/000661) describes an endoscope employing a flexible inflatable sleeve, which before inflation is retained within a dispenser. The dispenser employed in this endoscope has entry and exit ports defining a transit passage through which the endoscope may pass. The dispenser is adapted to capture the flexible sleeve as the endoscope is retracted through the transit passage in a proximal direction. In another embodiment, the dispenser includes an external sleeve fixed to the dispenser and this external sleeve is adapted to be extended from the dispenser when the endoscope is retracted so that the external sleeve covers the flexible sleeve. By virtue of this provision any contamination on the flexible sleeve remains within the external sleeve and does not contact the endoscope or any other objects or areas outside the patient's body. After the endoscope has been removed entirely from the flexible sleeve, the dispenser together with the external sleeve and the flexible sleeve is discarded.

It is mentioned in the above reference that the endoscope is provided with an internal sleeve, which is also known as multilumen tubing since it is usually fitted with appropriate passages or lumens as required for irrigation, ventilation, suction and for passing endoscopic tools therethrough. For operating the endoscope, the proximal end of the multilumen tubing is detachably connected via a dedicated disposable connector, or so called hub, to a source of fluid medium, i.e. water, compressed air and vacuum. A fluid control system is provided, which comprises an external control unit with a pump for supplying compressed air, a flask for supplying water and a pump for producing vacuum. The control unit is provided also with several pinch valves, which control the supply of compressed air, water and vacuum to the multilumen tubing and compressed air to the inflatable sleeve. The hub is detachably mounted on the frontal panel of the control unit and is fitted with the pinch valves. Through the hub pass flexible tubes for supplying fluid medium to the flexible sleeve and/or to the multilumen tubing.

Unfortunately maintenance of the endoscope provided with the above mentioned fluid control system is inconvenient and labor consuming, since before putting it into operation each tube should be connected one by one with a corresponding source of the fluid medium within the control unit.

The other disadvantage of the prior art control systems lies in the fact that it does not sufficiently prevent entrance of contaminated debris from the body channel back to the system.

There is also known a control system for supply of fluid medium to an endoscope as described in our patent application U.S. Ser. No. 60/608,432 herein incorporated by reference. The endoscope comprises an operation handle and an insertion tube provided with an insufflation channel, an irrigation channel and a suction channel. The control system is provided also with a system control unit comprising a pump, which supplies compressed air to the insufflation channel and to an inflatable flexible covering sleeve used with this endoscope. The system control unit also comprises electromagnetic normally opened valve, which upon receiving a signal opens the line for supplying compressed air to the sleeve. To prevent entrance of contamination in the system control unit through the sleeve, when the valve is closed, there is provided a trap means residing between the pump and the sleeve. This trap means comprises a spool valve, which is controlled by a control air. The main disadvantage of this solution is associated with the fact that the flow of air directed from the system control unit is available only in a situation when it is required to inflate the sleeve and not all the time. This renders preventing of the contamination entrance less reliable.

Furthermore, by virtue of this control system the sleeve is remote from the insertion tube only when it is inflated. In practice it would be desirable, however, that the sleeve would be always slightly inflated, since in this condition it is easier to displace the insertion tube backward and forward as might be required during the endoscopic procedure.

Still a further disadvantage of the above control system is associated with the fact that it does not allow controlling the flow rate of air supplied for inflation of the sleeve.

It should be borne in mind also that this control system requires a dedicated trap as well as the dedicated normally closed valve and a means for its control.

The main object of the present invention is to provide a new and improved control system and system control unit for supplying fluid medium to the multilumen tubing and/or to the inflatable sleeve of an endoscope provided with such a sleeve.

A further object of the invention is to provide a new and improved control system and system control unit, which is convenient and simple in operation and maintenance.

Still a further object of the invention is to provide a new and improved control system and system control unit, which always maintains flow of air from the system control unit to the sleeve, thus reliably preventing entrance of contamination from the body channel through the sleeve during the endoscopic procedure.

Another object of the invention is to provide a new and improved control system, which allows keeping the sleeve remote from the insertion tube irrespective whether the sleeve is being inflated or not.

Yet another object of the invention is to provide a new and improved control system and system control unit, which enables controlling of the flow rate of the air supplied for inflating the sleeve.

For a better understanding of the present invention as well of its benefits and advantages, reference will now be made to the following description of its embodiments taken in combination with the accompanying drawings. The main components of a modern gastroscopic apparatus comprise a flexible tube during the gastroscopic procedure.

### BRIEF DESCRIPTION OF THE DRAWINGS

FIG. 1 depicts a general view of an endoscopic apparatus employing the control system of the invention.

FIG.2 depicts an embodiment of the control system employed in the endoscopic apparatus of the invention.

### DETAILED DESCRIPTION OF THE INVENTION

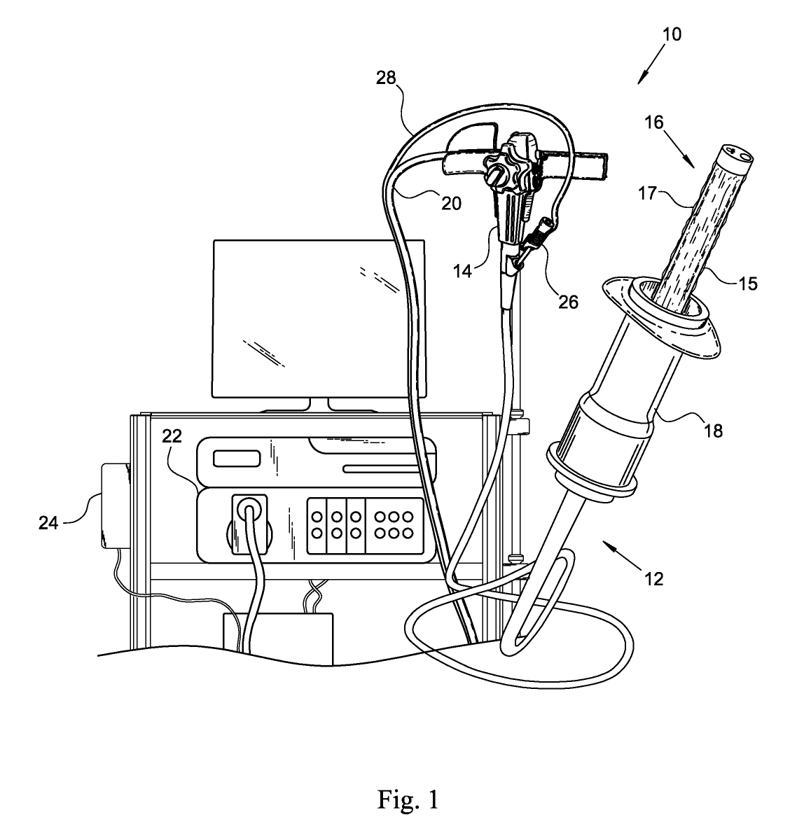

With reference to FIG. 1 an endoscopic, apparatus, preferably a colonoscopic apparatus 10, is shown with its following main components. The apparatus comprises an endoscope having insertion tube with its proximal section 12 connected to an operation handle 14 and with its distal section 16 inserted in and protruding from a disposable dispenser 18. An example of such an apparatus and general explanation of its construction and functioning can be found in Eizenfeld (WO 2004/016299; international application PCT/IL2003/000661). It is shown also in FIG. 1 that a disposable inflatable sleeve covers a distal region of the endoscope. That part of the sleeve, which is seen in FIG.1, comprises a frontal noninflatable portion 15 and a rear folded portion 17. The frontal portion covers the distal section of the endoscope and its head. The frontal portion does not inflate when the endoscope advances within the colon. The rear portion covers the insertion tube and unfolds when air or other fluid medium inflates the sleeve. By virtue of this provision the endoscope is propelled within the body passage. Explanation of this phenomenon can be found in the above referred-to reference. The endoscope, which can be used with the control system of the present invention, can be of similar type in the sense that it employs the same propelling mechanism, which is based on inflation of a flexible sleeve coupled to the endoscope's distal section. It should be appreciated, however, that the present invention is not limited merely to colonoscopy as such and to endoscopes, which are propelled by inflatable sleeve. It can be employed in any other endoscopic apparatus used for medical procedures requiring insertion of a probe in a body passage for inspection of its interior.

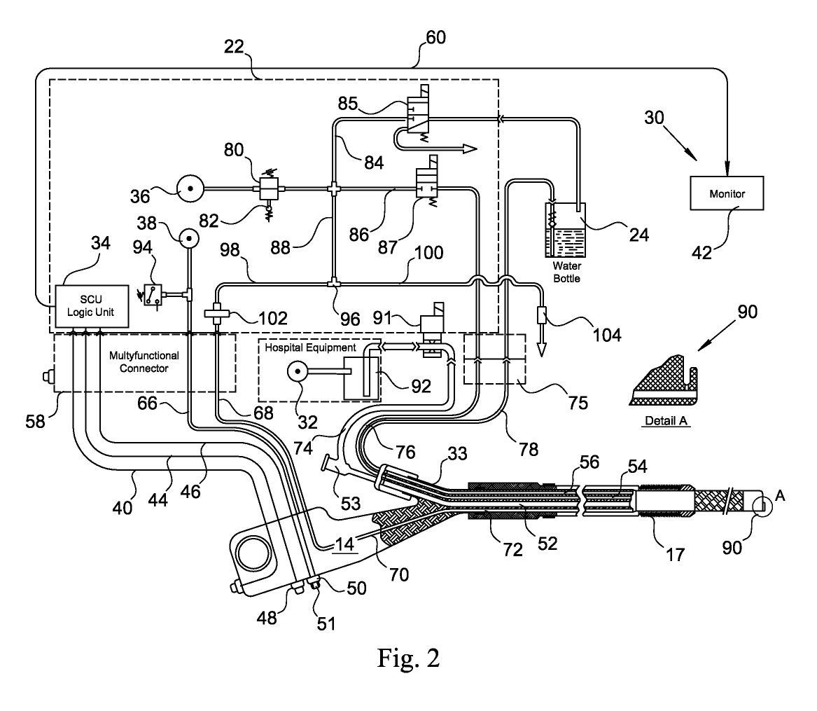

It is seen also in FIG. 1, that the handle is connected by an umbilical cord 20 to a system control unit (further referred-to as SCU) 22. Within the housing of the SCU there is provided a source of compressed air for inflating and venting the sleeve. Proximate to the system control unit a flask 24 is provided, which is filled with water, to be supplied under pressure into the colon for irrigation. It is not shown specifically, but should be appreciated that appropriate tubes extend along the umbilical cord for supplying air for insufflation and for supplying vacuum produced by appropriate means (not shown in FIG. 1). The SCU is one of the main parts of the control system and it will now be explained in more details in connection with FIG. 2. One should also bear in mind that within the insertion tube are provided various devices, which are necessary for functioning of the colonoscopic apparatus. These devices are known per se. Among such devices one can mention e.g. vertebrae and strings, which can be manipulated by the operation handle. It is not seen in FIG. 1, but should be appreciated, that along the colonsocope extends a multilumen tubing with appropriate passages for supplying water, as required for irrigation of the colon, air as required for insufflation and vacuum as required for suction. The multilumen tubing also is provided with a passage for introducing surgical instruments into the colon as might be required during the colonoscopic procedure. The multulumen tubing extends through the entire length of the endoscope, passes the handle and is connected to a dedicated Y-connector 26, which is detachably connectable to a lateral port provided on the handle so as to connect the proximal end of the multilumen tubing with tubes 28 extending along the umbilical cord and supplying water and air from the SCU to the multilumen tubing. In practice the multilumen tubing and the Y-connector are manufactured from plastic material. It would be advantageous if they are cheap, disposable items, which are discarded in the end of the endoscopic procedure after the colonoscope has been evacuated form the body passage. By virtue of this provision preparation to the new colonoscopic procedure is simple, convenient and fast and it is not associated with spreading of any contamination picked up from the body passage during the previous endoscopic procedure.

Referring now to FIG. 2 an embodiment of the fluid control system of the invention will be explained. The system is designated by reference numeral 30 and its main component, i.e. the SCU, is designated schematically by a dotted line. The SCU controls supply of air, water and vacuum as required for proper functioning of the colonoscopic apparatus 10. Some external components of the fluid control system, namely flask 24 and vacuum pump unit 32, are also seen. In practice the flask volume should be sufficient to contain about 300 cc of water. As a suitable source of vacuum one could use available hospital equipment capable of producing a vacuum of −0.4 bar to enable suction from the body passage through the multilumen tubing with a flow rate of at least 20 liter per minute. The multilumen tubing is seen in FIG. 2 and it is designated by reference numeral 33. Within the SCU are provided the necessary electronic, pneumatic and hydraulic components, e.g. a logic unit 34, a first and a second pump 36, 38 for supplying compressed air and various valves as will be explained later on. It is not shown specifically but should be appreciated that appropriate power supply means can be also provided within the SCU as required for activation of the valves and energizing the logic unit. In practice the first pump 36 should be capable of supplying air under pressure 0.5-0.7 bar with a flow rate 3-5 liters per minute. This pump is intended to supply compressed air to the multilumen tubing, to the inflatable sleeve and to the flask. The second pump 38 should be capable of supplying air under pressure 0.3 bar with a flow rate 2 liter per minute. This pump is intended for supplying air to the operating handle. The operating handle has an opening for releasing the air. The purpose of this arrangement will be explained further.

The logic unit is electrically connected by a line 40 to auxiliary control buttons provided on the handle. The auxiliary control buttons might be available for controlling a video signal supplied to a monitor 42, e.g. for saving or freezing the displayed picture. The logic unit is also electrically connected by two signal lines 44, 46 to respective control buttons 48 and 50 provided on the handle. Control button 48 enables controlling of suction through a channel 52 made in the multilumen tubing. This channel functions either as a suction channel (when vacuum is supplied therethrough) or as a working channel when it is required to insert a surgical tool through an external port 53. Control button 50 enables supply of air to the body passage through a dedicated insufflation channel 54. This control button also enables supply of water to the forwardmost end of the insertion tube through a dedicated irrigation channel 56. A through going opening 51 is provided in the control button 50. This opening can be closed or opened by the doctor's finger during operating of the handle. The through going opening is in flow communication with pump 38. For connecting the SCU with lines 40, 44 and 46 a detachable multifunctional connector 58 is provided. This connector is multifunctional in the sense that it enables not only passing electrical signals between the SCU and control buttons and auxiliary control buttons but also ensures supplying of compressed air to the handle.

The logic unit is electrically connected by a signal line 60 to monitor 42. In FIG. 2 are also seen tubes 66, 68, which provide flow communication between the SCU and the handle. The tubes are detachably connected to the SCU by virtue of multifunctional connector 58. It is seen that tube 66 serves for supplying pressurized air from pump 38 to opening 51 in the control button 50. It is also seen that tube 68 supplies pressurized air from pump 36 to the operating handle. Within the operating handle there is provided a passage 70 through which compressed air from pump 36 proceeds to a channel 72 extending through the insertion tube. This channel is used for inflating the sleeve. It is shown also in FIG. 2 that the multilumen tubing is in flow communication with the SCU via tubes 74, 76, 78, which are connected to the Y-connector provided at the lateral extension of the handle. These tubes respectively supply vacuum to the working channel 52, compressed air to the insufflation channel 54 and water to the irrigation channel 56. A common connector 75 is provided for simultaneous connecting tube 76 to the SCU and tube 78 to flask 24. In accordance with one of the aspects of the present invention tubes 76, 78 are immediately connectable and disconnectable to the respective source of air and water without the necessity to connect/disconnect the tubes one by one by separate connectors dedicated to each line. This provision renders the setting up of the control system very simple, convenient and fast.

Within the SCU are mounted various hydraulic and pneumatic components of the system, which are necessary for controlling supply of the fluid medium to the colonoscope. The fluid medium is supplied by the following supply lines: line a) for supplying of compressed air from pump 36 to the sleeve, to the multilumen tubing and to the flask; line b) for supplying vacuum produced by vacuum pump 32 to the multilumen tubing; line c) for supplying of compressed air from pump 38 to the handle; and line d) for supplying of water from flask 24 to the multilumen tubing. It is seen, for example, that in the line a) there is provided a pressure regulator 80 with a safety valve 82 for keeping the pressure supplied by pump 36 within a narrow range of 0.5-0.7 bar. The pressurized air proceeds via ducts 84, 86 to respective normally shut off solenoid valves 85, 87. These valves, when opened, enable supply of pressurized air from the pump either to flask 24 or to line 76. As soon as pressurized air is supplied to the flask, water within the flask is urged to proceed via supply tube 78 to the irrigation channel of the multilumen tubing so as to be ejected therefrom by means of a sprinkler means 90 provided at the forwardmost end of the insertion tube. In practice water is ejected from the flask with a flow rate of at least 1 cc per second. It can be readily appreciated that pressure is not permanently maintained within the flask, but only when it is required to supply water for irrigation. In the line b) there is provided a suction bottle 92 and a suction valve 91, which is a conventional pinch valve capable of selectively releasing the tube 74 passing therethrough. Pressing suction button 48 on the handle 14 actuates this pinch valve. It should be appreciated that all valves are electrically connected to the logic unit and are controlled thereby. In the line c) there is provided a pressure sensor 94, which senses air pressure in the line 66. The pressure sensor is electrically connected to the logic unit and as soon as the air pressure in line 66 reduces below a certain preset level, the sensor generates and sends to the logic unit a signal. Upon receiving this signal the logic unit opens valve SV1 and pressurized air is supplied via line 76 to the insufflation channel of the multilumen tubing.

It is also shown in FIG. 2 that a duct 88 is provided, through which compressed air is supplied from pump 36 via line 68 to passage 70. This duct splits at a knee 96 into a first branch 98 and a second branch 100. The first branch is in fluid communication via a filter 102 residing within line 68. The second branch terminates outside the SCU and is provided with a flow regulating means 104, through which air in the second branch can be released to the atmosphere. The flow regulating means is capable of varing the airflow rate in the second branch and to cause the air supplied by pump 36 to flow substantially either through the second branch or through the first branch. It can be readily appreciated that by virtue of this provision, pressurized air will always flow in one direction, namely from the system control unit to line 68 and channel 70. This directed flow prevents entrance of contamination through the sleeve to the system control unit when the sleeve is not inflated. In practice the flow regulating means can be any device capable of varying the flow rate by reducing the cross-sectional area of branch 100. An example of such a means would be a nozzle arranged at the operating handle and communicating with the atmosphere. If the nozzle is closed, e.g. by doctor's finger, the airflow from pump 36 will be directed to branch 98. To direct the airflow to branch 100 the doctor should remove his/her finger from the nozzle.

An alternative embodiment for the flow regulating means would be a foot pedal, which upon pressing is capable of elastically squeezing line 100 so as to reduce its cross-sectional area.

It would be beneficial, however, if the flow regulating means allows reducing the flow rate in a controllable fashion.

The control system depicted in FIG. 2 operates as follows. When it is not required to inflate the sleeve, the flow regulating means is set to release the airflow from pump 36 to atmosphere through branch 100. At the same time a fraction of the airflow will also pass from knee 96 to branch 98 and then via line 68 and channel 70 to the sleeve. This fractional airflow causes that although the sleeve is not inflated yet, it nevertheless will be remote from the outside surface of the insertion tube. By virtue of this provision displacing of the insertion tube along the body channel is easier and this improves the maneuverability of the insertion tube during the endoscopic procedure.

When it is required to inflate the sleeve, the flow regulating means is set to let the airflow to pass mostly through branch 98 and to inflate the sleeve. The possibility to change the flow rate in a controllable fashion renders the inflation of the sleeve also controllable, which, in its turn, renders the endoscopic procedure more convenient for the doctor and less painful for the patient.

In order to deflate the sleeve, one should set the flow regulating means again to let the airflow pass through branch 100 and to be released to the atmosphere. When suction is required one should press control button 48. Upon pressing this button, a signal will be generated by the logic unit to open pinch valve 91 and then vacuum will be established in suction channel 52. For insufflation one should close opening 51 provided on control button 50. Upon closure the pressure in line 66 increases, which will be detected by pressure sensor 94. Accordingly, the logic unit opens solenoid valve 87 and pressurized air will be allowed to proceed from pump 36 through tube 76 to the insufflation channel.

It should be borne in mind that instead of opening 51 closeable by doctor's finger as required for triggering of air supply from pump 38, one could use a different arrangement, e.g. electrical two stroke switch. To irrigate the body cavity one should deeply press control button 50. Then a signal will be generated by the logic unit to open valve 85. Upon opening the valve pressurized air is allowed to enter the flask and thus water is urged to proceed to the irrigation channel via tube 78. When there is no signal, the pressure is released from the valve immediately to the atmosphere and there is no pressure in the flask. It should be readily appreciated that control buttons 48, 50 are merely electrical switches, which are electrically connected to the logic unit and there is no flow communication between the buttons and the multilumen tubing. By virtue of this provision the danger of contaminating the buttons by any debris penetrating from the body channel or cavity is prevented. At the same time, since flow of air is permanently maintained in the direction from pump 38 to opening 51, it is possible to retain the same mode of carrying out the insufflation or irrigation, to which the doctors are so accustomed. In accordance with this mode a finger pressure on the hole in the center of button 50 provides insufflation and further depression of the button triggers the irrigation.

It should be also appreciated that since the control buttons operate the valves electrically and not mechanically there is no need in mechanical parts, like pistons etc. In the prior art systems the control buttons usually are associated with a mechanical control mechanism comprising mechanical parts. Due to inevitable contamination it is required to dismantle the control mechanism and to clean it after each colonoscopy session. In the present invention the buttons are not associated with any mechanical parts, which could get contaminated. It should be appreciated that the invention is not limited to the above-described embodiments and that one ordinarily skilled in the art can make modifications or changes without deviating from the scope of the invention, as will be defined in the appended claims. It should also be appreciated that the features disclosed in the foregoing description, and/or in the following claims, and/or in the accompanying drawings may, both separately and in any combination thereof, be material for realizing the present invention in diverse forms thereof.

We claim:

1. A control system for supplying a fluid medium to an endoscopic apparatus comprising an operation handle and an insertion member insertable within a body channel, wherein there is provided a protection sleeve covering at least a portion of the insertion member, said insertion member being provided with extending therealong an insufflation channel, an irrigation channel, a suction channel and a channel for inflating the sleeve, said control system comprising:
a) a system control unit comprising a pump for supplying compressed air at least to the channel for inflating the sleeve, pneumatic and hydraulic components for facilitating fluid transmission, a logic unit for controlling said pump and said hydraulic and pneumatic components, and a duct in fluid communication with the pump, said duct splitting to a first branch through which the compressed air flows from the pump to the sleeve and to a second branch having exit to atmosphere, said second branch being provided with a flow regulating means to admit the compressed air from the pump either substantially to the sleeve or to the atmosphere,
b) a source of liquid in fluid communication with the irrigation channel, and
c) a source of vacuum in fluid communication with the suction channel.

2. The control system as defined in claim 1, wherein said flow regulating means changes the pressure in the second branch in a controllable fashion.

3. The control system as defined in claim 1, wherein said flow regulating means comprises a nozzle.

4. The control system as defined in claim 3, wherein said nozzle is arranged at the operation handle.

5. The control system as defined in claim 4, wherein said nozzle is closeable by the operator's finger.

6. The control system as defined in claim 1, wherein said flow regulating means comprises a foot pedal, wherein upon pressing by the operator's foot elastically squeezes at least a portion of the second branch.
